(19) United States
(12) Patent Application Publication
(10) Pub. No.: US 2011/0184518 A1
(43) Pub. Date: Jul. 28, 2011

(54) SACRO-ILIAC JOINT IMPLANT

(75) Inventor: Hai H. Trieu, Memphis, TN (US)

(73) Assignee: WARSAW ORTHOPEDIC, INC., Warsaw, IN (US)

(21) Appl. No.: 12/692,331

(22) Filed: Jan. 22, 2010

(51) Int. Cl. A61F 2/44

(52) U.S. Cl. 623/17.11

(57) ABSTRACT

A sacro-iliac implant includes at least one wedge-shaped body. The body defines an outer surface configured to engage at least one articular surface of a sacro-iliac joint. Systems and methods employing the sacro-iliac implant are disclosed.

### TECHNICAL FIELD

The present disclosure generally relates to medical devices for the treatment of musculoskeletal disorders, and more particularly to an implant system and method for treating the sacro-iliac joint.

### BACKGROUND

The sacroiliac (SI) joint is a diarthrodial joint that joins the sacrum to the ilium bones of the pelvis. In the SI joint, the sacral surface has hyaline cartilage that moves against fibrocartilage of the iliac surface. The spinal column is configured so that the weight of an upper body rests on the SI joints at the juncture of the sacrum and ilia. Stress placed on the SI joints in an upright position of the body makes the lower back susceptible to injury.

Disorders of the SI joint can cause low back and radiating buttock and leg pain in patients suffering from degeneration and laxity of the SI joint. In some cases, the SI joint can undergo dehydration and destabilization, similar to other cartilaginous joints, which causes significant pain. The SI joint is also susceptible to trauma and degeneration, from fracture and instability. It is estimated that disorders of the SI joint are a source of pain for millions of people suffering from back and radicular symptoms.

Non-surgical treatments, such as medication, injection, mobilization, rehabilitation and exercise can be effective, however, may fail to relieve the symptoms associated with these disorders. Surgical treatment of these disorders includes stabilization and/or arthrodesis. Stabilization can include the use of bone screws that are directly threaded into bone. Arthrodesis may include fusion devices to immobilize a joint. The present disclosure describes an improvement over these prior art technologies.

### SUMMARY OF THE INVENTION

Accordingly, an implant system and method is provided for treating the SI joint. It is contemplated that the system may include an implant configured for disposal with the SI joint. It is further contemplated that the implant system and method may be employed for arthroplasty treatment.

In one particular embodiment, in accordance with the principles of the present disclosure, a sacro-iliac implant is provided. The sacro-iliac implant includes at least one wedge-shaped body defining an outer surface configured to engage at least one articular surface of a sacro-iliac joint.

In one embodiment, an orthopedic implant includes at least one wedge shaped body defining an outer surface configured to engage a first articular surface and a second opposing articular surface. The outer surface includes a first side surface and a second side surface. The body includes a pointed tip and defines at least one channel. The channel extends between the side surfaces and defines at least one first opening disposed with the body in an orientation facing the first articular surface. At least one second opening is disposed with the body in an orientation facing the second articular surface.

In one embodiment, a sacro-iliac implant includes at least one wedge shaped body defining an outer surface configured to engage a first articular surface and a second opposing articular surface. The outer surface includes a first side surface and a second side surface. The body includes a pointed tip and defines at least one channel extending between the side surfaces. The body defines at least one first opening disposed with the body in an orientation facing the first articular surface and at least one second opening disposed with the body in an orientation facing the second articular surface. The at least one channel including an agent reservoir disposed between the openings and the at least one channel being configured to expel and/or elute at least one agent from the openings.

### BRIEF DESCRIPTION OF THE DRAWINGS

The present disclosure will become more readily apparent from the specific description accompanied by the following drawings, in which:

FIG. 1 is a perspective view of one particular embodiment of an implant of an implant system in accordance with the principles of the present disclosure;

FIG. 2 is a plan view of the implant shown in FIG. 1 of an implant system and a sacro-iliac/ilio-pelvic region;

FIG. 3 is a plan view of the implant system and an SI joint of the region shown in FIG. 2;

FIG. 4 is a plan view of the implant system and the SI joint shown in FIG. 2;

FIG. 5 is a plan view of the implant system and the SI joint shown in FIG. 2;

FIG. 6 is a perspective view of one embodiment of an implant of the implant system shown in FIG. 1;

FIG. 7 is a perspective view of one embodiment of an implant of the implant system shown in FIG. 1;

FIG. 8 is a perspective view of one embodiment of an implant of the implant system shown in FIG. 1;

FIG. 9 is a perspective view of one embodiment of an implant of the implant system shown in FIG. 1;

FIG. 10 is a perspective view of one embodiment of an implant of the implant system shown in FIG. 1;

FIG. 11 is a perspective view of one embodiment of an implant of the implant system shown in FIG. 1;

FIG. 12 is a perspective view of one embodiment of an implant of the implant system shown in FIG. 1;

FIG. 13 is a perspective view of one embodiment of an implant of the implant system shown in FIG. 1;

FIG. 14 is a perspective view of one embodiment of an implant of the implant system shown in FIG. 1;

FIG. 15 is a perspective view of one embodiment of an implant of the implant system shown in FIG. 1; and

FIG. 16 is a perspective view of one embodiment of an implant of the implant system shown in FIG. 1.

Like reference numerals indicate similar parts throughout the figures.

### DETAILED DESCRIPTION OF THE INVENTION

The exemplary embodiments of the implant system and methods of use disclosed are discussed in terms of medical devices for the treatment of musculoskeletal disorders and more particularly, in terms of an implant system and method for treating the SI joint. It is envisioned that the implant system and methods of use disclosed provide stability and maintains structural integrity while reducing stress on the SI joint. It is further envisioned that the present disclosure may be employed to treat musculoskeletal disorders including sacro-Iliac dysfunction or syndrome, dehydration, destabilization, laxity, fracture, tumor, spinal disorders and other orthopedic disorders. It is contemplated that the present disclosure may be employed with surgical treatments, including open surgery, percutaneous and minimally invasive procedures of such disorders, such as, for example, arthroplasty to maintain motion and implantable prosthetics. It is further contemplated that the present disclosure may be employed with other osteal and bone related applications, including those associated with diagnostics, motion preservation and therapeutics. The disclosed implant system and methods may be employed in a surgical treatment with a patient in a prone or supine position, employing a posterior, lateral, inferior, posterior-inferior, superior or anterior approach. The present disclosure may be employed with procedures for treating the lumbar, cervical, thoracic and pelvic regions of a spinal column.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

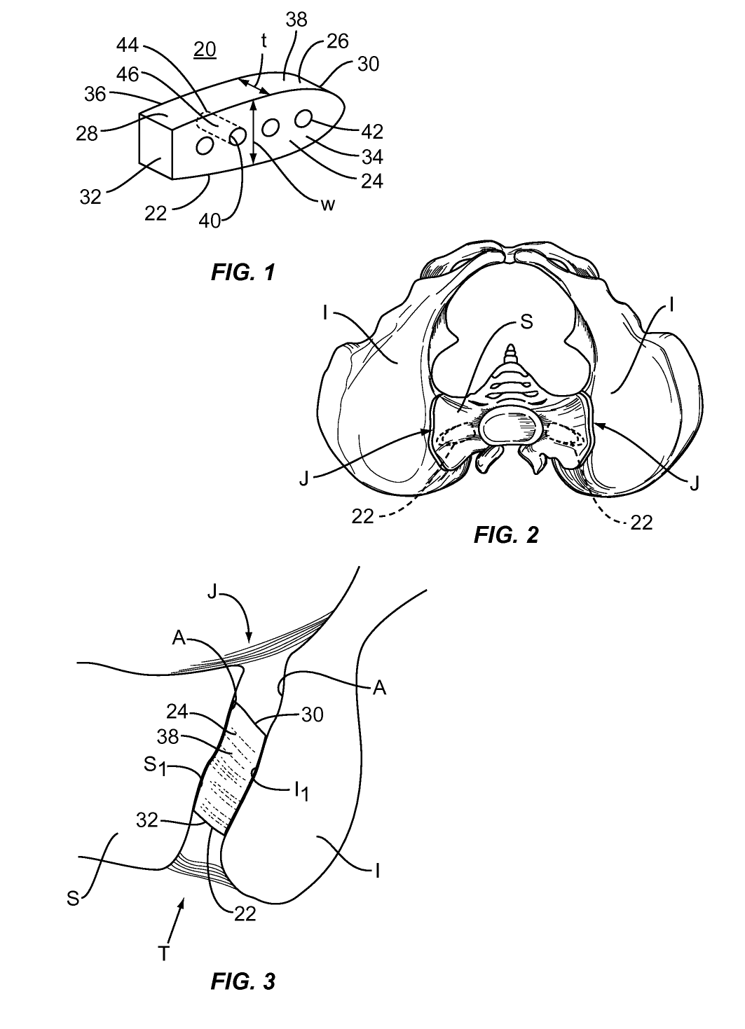

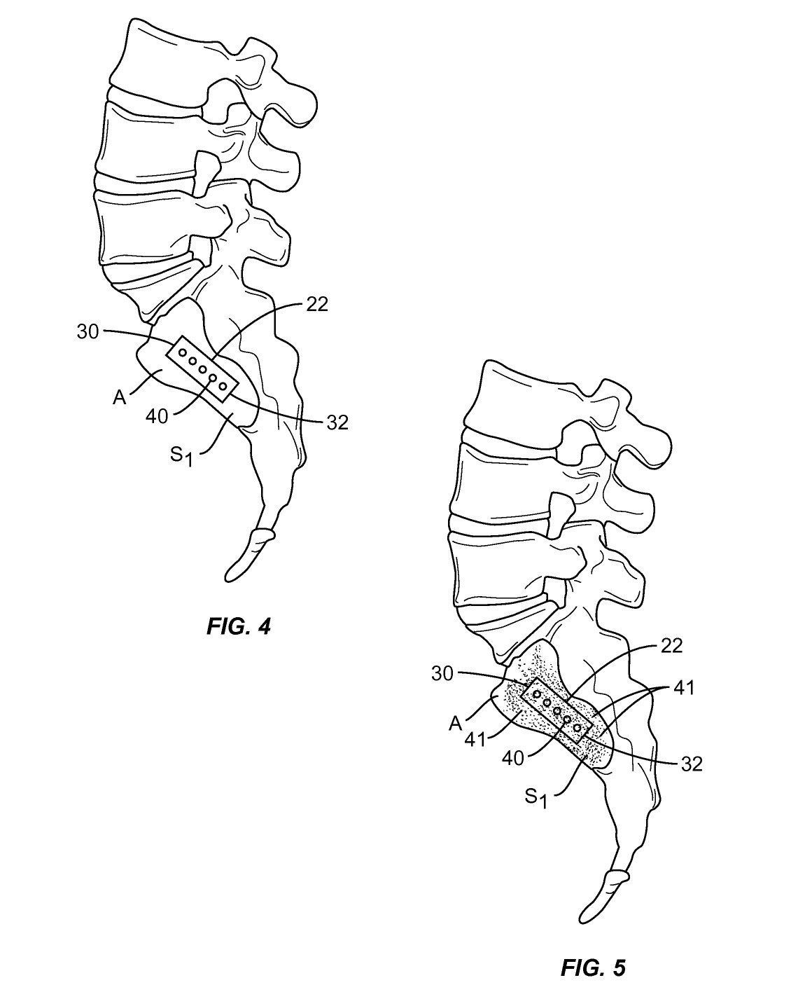

The following discussion includes a description of an implant system, related components and exemplary methods of employing the implant system in accordance with the principles of the present disclosure. Alternate embodiments are also disclosed. Reference will now be made in detail to the exemplary embodiments of the present disclosure, which are illustrated in the accompanying figures. Turning now to FIGS. 1-5, there are illustrated components of the implant system in accordance with the principles of the present disclosure.

The components of the implant system are fabricated from materials suitable for medical applications, including metals, synthetic polymers, ceramics, bone, biocompatible materials and/or their composites, depending on the particular application and/or preference of a medical practitioner. For example, components of the implant system, such as, for example, a wedge-shaped body, an outer surface of the wedge-shaped body and/or portions thereof, and/or channels of the body, which may be monolithically formed, integrally connected or configured as an insert with the body, discussed below, can be fabricated from materials such as commercially pure titanium, titanium alloys, super-elastic titanium alloys, cobalt-chrome alloys, stainless steel alloys, thermoplastics such as polyaryletherketone (PAEK) including polyetheretherketone (PEEK), polyetherketoneketone (PEKK) and polyetherketone (PEK), carbon fiber reinforced PEEK composites, PEEK-BaSO₄ composites, ceramics and composites thereof, rigid polymers including polyphenylene, polyamide, polyimide, polyetherimide, polyethylene, polyurethanes of any durometer, epoxy, silicone, bone material including autograft, allograft, xenograft or transgenic cortical and/or corticocancellous bone, and tissue growth or differentiation factors. Different components of the implant system may have alternative material composites to achieve various desired characteristics such as strength, rigidity, elasticity, compliance, biomechanical performance, durability and radiolucency or imaging preference. The individual components of the implant system may also be fabricated from a heterogeneous material such as a combination of two or more of the above-described materials.

It is envisioned that the components of the implant system can be manufactured via various methods. For example, the wedge-shaped body can be manufactured and assembled via injection-molding, insert-molding, overmolding, compression molding, transfer molding, co-extrusion, pultrusion, dip-coating, spray-coating, powder-coating, porous-coating, milling from a solid stock material, and their combinations. One skilled in the art, however, will realize that such materials and fabrication methods suitable for assembly and manufacture, in accordance with the present disclosure, would be appropriate.

The implant system includes an orthopedic implant, such as, for example, a sacro-iliac implant 20, which is configured, for example, to treat S-I joint disorders including those caused by degeneration or trauma. It is contemplated that sacro-iliac implant 20 may be employed for arthroplasty applications, as will be described.

Sacro-iliac implant 20 includes a wedge-shaped body 22 that defines an outer surface 24. Outer surface 24 is configured to engage an articular surface A of a sacro-iliac joint J. It is contemplated that articular surface A may refer to a sacral surface S₁ of a sacrum S and/or an iliac surface I₁ of an ilium I. Body 22 may be configured to engage opposing articular surfaces such as sacral surface S₁ and iliac surface I₁ and/or opposing valleys or peaks of an individual sacrum S or ilium I. Body 22 may have a solid, hollow, porous or cage configuration. Outer surface 24 has a continuously even or smooth configuration. It is contemplated that outer surface 24 is configured to substantially match articular surface(s) A and may be substantially smooth, rough, textured, dimpled and/or polished.

Body 22 extends from a first end 26 to a second end 28. First end 26 includes a pointed tip 30. Pointed tip 30 is configured, for example, for facile insertion with a narrow joint such as a SI joint and/or for minimally invasive applications. Wedge shape body 22 gradually increases or tapers from tip 30 in width from first end 26 to second end 28, which includes a base 32. This wedge configuration of body 22 facilitates disposal of body 22 within an SI joint, such that, for example, body 22 separates articular surfaces A to eliminate pain by dilating the SI joint and preventing joint surfaces from undesired engagement such as that caused by degeneration and cartilage wear, and may also facilitate tensioning of ligaments. Body 22 has a thickness t and a width w, according to the requirements of the particular application. It is envisioned that thickness t may be in a range of approximately 1-12 millimeters (mm), and preferably in a range of approximately 3-9 millimeters. It is contemplated that width w may be in a range of approximately 5-30 mm, and preferably in a range of approximately 10-25 mm. It is further contemplated that the cross-sectional geometry of body 22 may have various configurations, for example, round, oval, rectangular, polygonal, irregular, uniform, non-uniform, consistent or variable.

Body 22 has a first load surface 34 and an opposing second load surface 36 that extend along width w, which tapers from first end 26 to second end 28. Outer surface 24 includes adjacent side surfaces 38, extending between first side surface 34 and second side surface 36, along thickness t. Each of load surfaces 34, 36 has a surface area of at least greater than approximately 1.5 to 30 times, and preferably approximately 3 to 15 times a surface area of a side surface 38. The greater surface area of load surfaces 34, 36 relative to each side surface 38 provides support of articular surfaces A thereby facilitating improved joint dilation. It is envisioned that thickness t may be unequal along surface 34, 36 such that surface 34, 36 may be offset, tapered, converging and/or diverging.

It is envisioned that body 22 can be variously configured and dimensioned with regard to size, shape, thickness, geometry and material. Body 22 may also be formed of one or a plurality of elements such as spaced apart portions, staggered patterns and mesh. It is envisioned that the particular geometry and material parameters of body 22 may be selected to modulate the flexibility or stiffness of sacro-iliac implant 20, such as those examples discussed herein. For example, body 22 can be configured to have varying ranges or degrees of flexibility or stiffness such as rigid, compliant, or reinforced. Depending on the flexibility or stiffness of body 22, the flexibility or stiffness of sacro-iliac implant 20 can be contoured according to the requirements of a particular application. It is contemplated that the ability to vary stiffness of sacro-iliac implant 20 provides restoration of kinematic function of joint J. It is envisioned that the components of sacro-iliac implant 20 may be monolithically formed, integrally connected or arranged with attaching elements.

Body 22 defines a plurality of cavities, such as, for example, channels 40. Channels 40 are configured to expel and/or elute at least one agent 41 therefrom. Each of channels 40 define an opening 42 in first load surface 34 and an opening 44 in second load surface 36. Channel 40 includes an agent reservoir 46 disposed between openings 42, 44. It is contemplated reservoir(s) 46 is disposed below outer surface 24 and in fluid communication with tissues engaging outer surface 24 via openings 42, 44. It is further contemplated that reservoir(s) 46 fluidly communicate with tissues engaging outer surface 24 via a series of pores formed in body 22 and/or body 22 formed of a porous material. In one embodiment, body 22 includes multiple reservoirs that facilitate different release rates for agent(s) disposed therein. Such multiple reservoirs may or may not be in fluid communication. It is envisioned that openings 42, 44 may be oriented parallel to articular surface(s) A in a configuration to expel or elute at least one agent through surfaces 34, 36 into a SI joint.

Opening 42 is disposed with body 22 in an orientation facing a first articular surface A, such as, for example, sacral surface S₁, and opening 44 is disposed with body 22 in an orientation facing a second articular surface A, such as, for example, iliac surface I₁. Channel 40 can include reservoirs such as drug depots with medication for pain and may include antibiotics and/or therapeutics. Diffusion of such agents can occur through channels 40. It is contemplated that the channels and/or reservoirs can be configured for multiple directional expulsion and/or eluting. For example, the channels and/or reservoirs can be oriented toward joint surfaces, perpendicular to joint surfaces, parallel to joint surfaces or randomly oriented.

In one embodiment, body 22 includes channels 40, which include a network of channels 40 (not shown) interconnected and/or in fluid communication. It is envisioned that selected channels 40 may include portions or sub-cavities within body 22 that are not directly communicating with openings 42, 44. It is further envisioned that body 22 may include one or a plurality of channels. It is contemplated that body 22 and/or each of channels 40 may include one or a plurality of agents. It is envisioned that channels 40 may have a diameter in a range of approximately 10-1,000 microns, and preferably in a range of approximately 100-500 microns. It is further envisioned that channels 40 may interconnect to form a microporous body 22.

In one embodiment, the implant system includes a plurality of wedge-shaped bodies 22, described above. It is contemplated that employing the plurality of bodies 22 can optimize the amount joint J can be spaced apart such that a joint space JS dimension can be preselected. The plurality of bodies 22 can be inserted through the same or an alternate trajectory. The plurality of bodies 22 can be oriented in a side by side engagement, spaced apart and/or staggered. It is envisioned that one or all of the plurality of bodies 22 may be inserted via a trajectory oriented from an anterior, posterior, superior or inferior direction, similar to that described with regard to FIG. 3. It is further envisioned that one or a plurality of bodies 22 may be used.

In assembly, operation and use, the implant system including sacro-iliac 20 is employed with a surgical procedure for treatment of a sacro-iliac joint J of a patient, as discussed herein. The implant system may also be employed with other surgical procedures. In particular, the implant system is employed with a surgical arthroplasty procedure for treatment of an applicable condition or injury of an affected sacro-iliac joint J, as shown in FIGS. 2-5. It is contemplated that the implant system is inserted with a sacro-iliac joint to provide a less invasive approach for treatment and re-establish joint tension. It is further contemplated that the implant system is inserted with a sacro-iliac joint as a SI joint spacer to restore ligamentous tension, eliminate painful micro-motion, separate and cushion opposing articulating surfaces that cause pain and/or preserve motion. It is envisioned that the implant system may maintain joint tension without promoting bone growth.

In use, to treat the affected section of sacro-iliac joint J, a medical practitioner obtains access to a surgical site including sacro-iliac joint J in any appropriate manner, such as through incision and retraction of tissues. It is envisioned that the implant system may be used in any existing surgical method or technique including open surgery, mini-open surgery, minimally invasive surgery and percutaneous surgical implantation, whereby sacro-iliac joint J is accessed through a mini-incision, or sleeve that provides a protected passageway to the area. Once access to the surgical site is obtained, the particular surgical procedure is performed for treating the sacro-iliac joint disorder. The implant system is then employed to augment the surgical treatment. The implant system can be delivered or implanted as a pre-assembled device or can be assembled in situ. The implant system may be completely or partially revised, removed or replaced in situ.

A trajectory T, as shown in FIG. 3, is defined for insertion and/or injection of sacro-iliac implant 20 within sacro-iliac joint J. Implant 20 is inserted via the protected passageway along the defined trajectory T into sacro-iliac joint J. A cavity of sacro-iliac joint J is prepared along trajectory T for disposal of sacro-iliac implant 20.

The protected passageway includes a dilator/delivery tube (not shown) configured to deliver sacro-iliac implant 20 directly to joint space JS of sacro-iliac joint J. It is envisioned that the dilator/delivery tube may be configured as an in-situ guidable instrument, and may include an endoscope camera tip for viewing insertion trajectory.

Sacro-iliac implant 20 is manipulated such that load surfaces 34, 36 of body 22 engage opposing articular surfaces A, according to the contour of articular surfaces A. Manipulation can include pushing, pulling, rotation of sacro-iliac implant 20, rotation of sacro-iliac implant 20 about the joint axis once implanted and/or by mechanical devices. It is contemplated that body 22 may engage only one or a plurality of articular surfaces A.

Sacro-iliac implant 20 is disposed with sacro-iliac joint J for treating the sacro-iliac joint disorder. Body 22 is configured for compliant engagement with articular surfaces A. Body 22 engages articular surface A and is secured within joint JS to allow relative motion of the articular surfaces A of the sacrum and ilium of sacro-iliac joint J.

Openings 42, 44 are oriented to face articular surfaces A, such as sacral surface S₁ and iliac surface I₁ such that at least one agent 41, as shown in FIG. 5, are expelled and/or eluted from channels 40. It is envisioned that reservoir 46 contains active agents 41. It is further envisioned that agent 41 can include one or a plurality of therapeutic agents and/or pharmacological agents for release, including sustained release, into SI joint J to treat, for example, pain, inflammation and degeneration. Agent 41 may include pharmacological agents, such as, for example, antibiotics, pain medications, analgesics, anesthetics, anti-inflammatory drugs including but not limited to steroids, anti-viral and anti-retroviral compounds, therapeutic proteins or peptides, therapeutic nucleic acids (as naked plasmid or a component of an integrating or non-integrating gene therapy vector system), and combinations thereof.

Agent 41 may also include analgesics or anesthetics such as acetic acid derivatives, clonidine, COX-2 selective inhibitors, COX-2 inhibitors, enolic acid derivatives, propionic acid derivatives, salicylic acid derivatives, opioids, opioid/nonopioid combination products, adjuvant analgesics, and general and regional/local anesthetics.

Agent 41 may include antibiotics such as, for example, amoxicillin, beta-lactamases, aminoglycosides, beta-lactam (glycopeptide), clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamthoxazole, and vancomycin.

Agent 41 may include immunosuppressives agents, such as, for example, steroids, cyclosporine, cyclosporine analogs, cyclophosphamide, methylprednisone, prednisone, azathioprine, FK-506, 15-deoxyspergualin, prednisolone, methotrexate, thalidomide, methoxsalen, rapamycin, leflunomide, mizoribine (Bredinin™), brequinar, deoxyspergualin, and azaspirane (SKF 105685), Orthoclone OKT™ 3 (muromonab-CD3). Sandimmune™, Neoral™, Sangdya™ (cyclosporine), Prograf™ (FK506, tacrolimus), Cellcept™ (mycophenolate motefil, of which the active metabolite is mycophenolic acid), Imuran™ (azathioprine), glucocorticosteroids, adrenocortical steroids such as Deltasone™ (prednisone) and Hydeltrasol™ (prednisolone), Folex™ and Mexate™ (methotrxate), Oxsoralen-Ultra™ (methoxsalen) and Rapamuen™ (sirolimus).

Channels 40 are capable of accepting at least one agent 41 before, during and/or after implantation with joint J, holding the at least one agent 41 in reservoir 46, and/or delivery in vivo of the at least one agent 41 to tissues of joint J and tissues surrounding joint J, including bone. Channels 40 may be replenished, via one or a plurality of iterations, with therapeutic and/or pharmacological agents.

The at least one agent 41 may be eluted from channels 40 through openings 42, 44 via an unstressed, free-flowing fluid communication with joint tissues, including bone, engaging outer surface 24. The at least one agent 41 may also be expelled from channels 40 through openings 42, 44 via compression of body 22 between articular surfaces A such that the at least one agent 41 is expelled or squeezed therefrom. It is contemplated that fabrication of body 22 with a permeable material facilitates permeation and diffusion of the at least one agent 41 through the material of body 22 into channels 40 and/or directly into the SI joint.

Outer surface 24 of body 22 may be compressible. It is envisioned that body 22 may be inserted via a trajectory oriented from a posterior, lateral, inferior, posterior-inferior, superior or anterior direction.

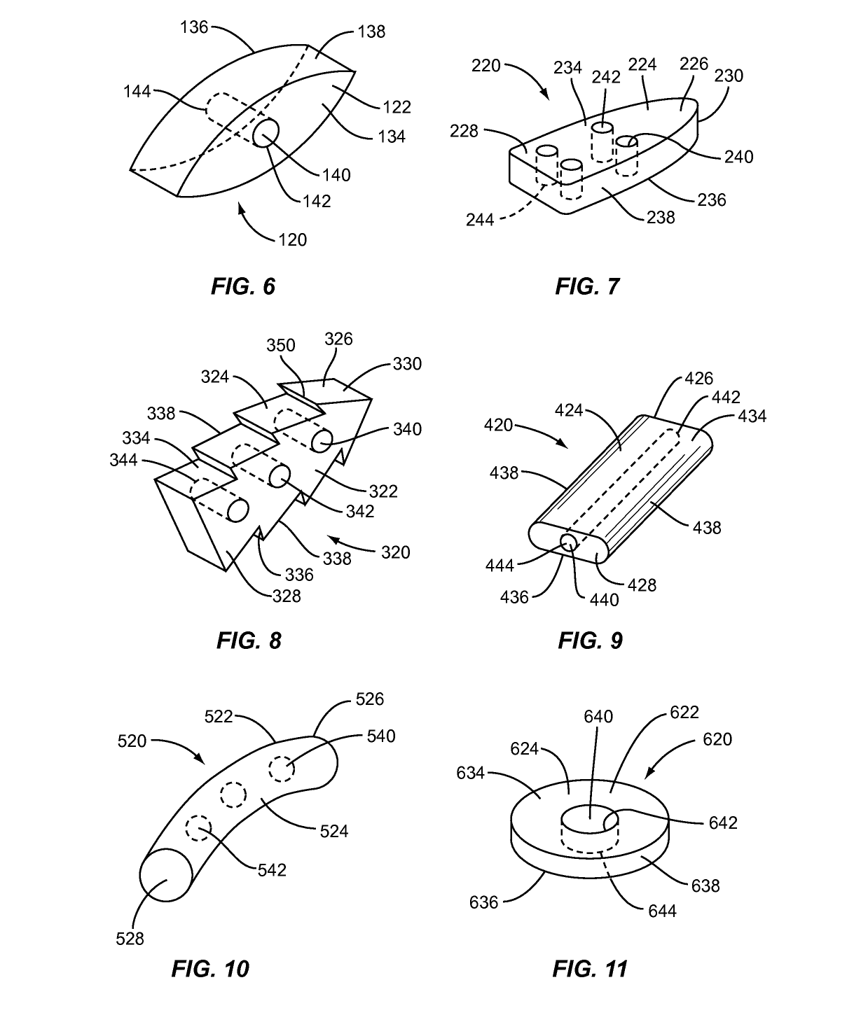

Referring to FIG. 6, in one embodiment similar to implant 20 and the implant system described above, a sacro-iliac implant 120 is configured, for example, to treat S-I joint disorders. Implant 120 has a body 122 with a wide load surface 138 having a greater surface area relative to side surfaces 134, 136, which provides improved support. Load surface 138 is configured to engage articular surface(s) A, similar to that described above. Body 122 has a single channel 140, similar to channel 40 described above, for expelling and/or eluting at least one agent therefrom. Channel 140 defines an opening 142 in side surface 134 and an opening 144 in side surface 136.

Referring to FIG. 7, in one embodiment similar to implant 20 and the implant system described above, a sacro-iliac implant 220 is configured, for example, to treat S-I joint disorders. Sacro-iliac implant 220 includes a body 222 extending from a first end 226 to a second end 228, and includes a pointed tip 230. Body 222 has an outer surface 224, which includes a first load surface 234 and a second load surface 236, and adjacent side surfaces 238. Load surfaces 234, 236 each have a wider load surface and a greater surface area relative to side surfaces 238. Load surfaces 234, 236 are configured to engage articular surface(s) of an SI joint, similar to that described above. Body 222 includes a plurality of channels 240, similar to those described above, for expelling and/or eluting at least one agent therefrom. Channels 240 define openings 242 within load surface 234 and openings 244 within load surface 236. It is envisioned that body 222 may include one or a plurality of channels 240.

Referring to FIG. 8, in one embodiment similar to implant 20 and the implant system described above, a sacro-iliac implant 320 is configured, for example, to treat S-I joint disorders. Sacro-iliac implant 320 includes a body 322 having an elongated configuration and extending from a first end 326 to a second end 328. Body 322 has a pointed tip 330. Body 322 has an outer surface 324, which includes a first load surface 334 and a second load surface 336, and adjacent side surfaces 338. Body 322 includes a locking element, such as, for example, barbs 350 disposed along opposing surfaces 334, 336, which are configured to engage articular surface(s) of an SI joint, similar to that described above, and prevent disengagement therefrom. Opposing surfaces 338 have a flat configuration. Body 322 includes a plurality of channels 340, similar to that described, for expelling and/or eluting at least one agent therefrom. Channels 340 define openings 342, 344 in opposing surfaces 338. Body 322 may include one or a plurality of barbs 350. Body 322 may include one or a plurality of channels 340.

Referring to FIG. 9, in one embodiment similar to implant 20 and the implant system described above, a sacro-iliac implant 420 is configured, for example, to treat S-I joint disorders. Sacro-iliac implant 420 includes a body 422 having an elongated configuration and extending from a first end 426 to a second end 428, which are planar in configuration. Body 422 has an outer surface 424, which includes elongated, planar load surfaces 434, 436, and arcuate side surfaces 438. This configuration of body 422 provides a thin, elongated implant 420 that can be easily inserted with a joint space. Body 422 defines an elongated longitudinal channel 440, similar to that described above, for expelling and/or eluting at least one agent therefrom. Channel 440 defines an opening 442 in first end 426 and an opening 444 in second end 428. Body 422 may include one or a plurality of channels 440 disposed in transverse and angled orientations.

Referring to FIG. 10, in one embodiment similar to implant 20 and the implant system described above, a sacro-iliac implant 520 is configured, for example, to treat S-I joint disorders. Sacro-iliac implant 520 includes a body 522 having an elongated, tubular configuration with a curvature, according to the requirements of a particular application. Body 522 extends from a first end 526 to a second end 528, which have a planar configuration. Body 522 has a circumferential outer surface 524, which is smooth and/or even. Surface 524 may have alternate configurations such as undulating, textured and/or dimpled. Body 522 includes a plurality of channels 540, similar to that described, for expelling and/or eluting at least one agent therefrom. Channels 540, which extend through body 522, define openings 542. Body 522 may include one or a plurality of channels 540.

Referring to FIG. 11, in one embodiment similar to implant 20 and the implant system described above, a sacro-iliac implant 620 is configured, for example, to treat S-I joint disorders. Sacro-iliac implant 620 includes a body 622 having a thin, disc configuration that facilitates easy insertion with a joint space. Body 622 has an outer surface 624, which includes opposing, cylindrical load surfaces 634, 636, and a circumferential, side surface 638. Body 622 includes a channel 640, similar to that described, for expelling and/or eluting at least one agent therefrom. Channel 640 defines an opening 642 in surface 634 and an opening 644 in surface 636.

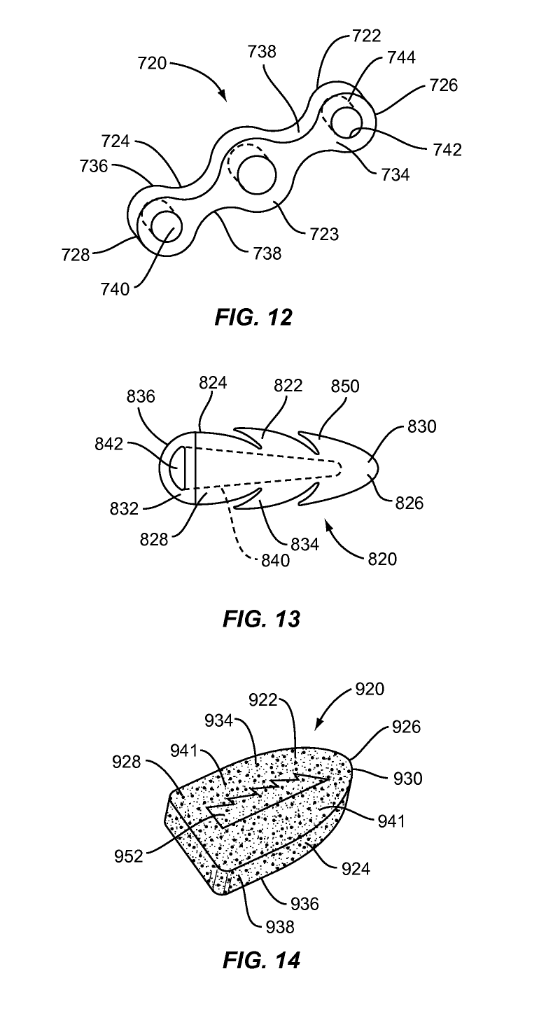

Referring to FIG. 12, in one embodiment similar to implant 20 and the implant system described above, a sacro-iliac implant 720 is configured, for example, to treat S-I joint disorders. Sacro-iliac implant 720 includes a body 722 having an elongated, link configuration including connected links 723. Body 722 extends from a first end 726 to a second end 728. Body 722 has an outer surface 724, which includes planar load surfaces 734, 736, and an undulating side surface 738. Each link 723 includes a channel 740, similar to that described, for expelling and/or eluting at least one agent therefrom. Channels 740 include openings 742 defined in surface 734 and openings 744 defined in surface 736.

Referring to FIG. 13, in one embodiment similar to implant 20 and the implant system described above, a sacro-iliac implant 820 is configured, for example, to treat S-I joint disorders. Sacro-iliac implant 820 includes a body 822 having an elongated, tubular configuration. Body 822 has a half cylinder shape and is deformable for easy insertion with a joint space. Body 822 extends from a first end 826 to a second end 828. First end 826 includes a pointed tip 830 and second end 828 has a planar base 832. Body 822 has an outer surface 824, which includes a planar load surface 834 and an arcuate load surface 836. Load surface 836 includes a locking element, such as, for example, barbs 850, similar to barbs 350 described with regard to FIG. 8. Body 822 includes an elongated channel 840, which is closed at first end 826 and defines an opening 842 in second end 828, for expelling and/or eluting at least one agent therefrom.

Referring to FIG. 14, in one embodiment similar to implant 20 and the implant system described above, a sacro-iliac implant 920 is configured, for example, to treat S-I joint disorders. Sacro-iliac implant 920 includes a body 922 having an agent 941, similar to those described, homogeneously distributed thereabout. Agent 941 is eluted to tissues, which may include bone, surrounding implant 920. It is contemplated that agent 941 may be an active agent, such as, for example, NSAIDS, steroids, analgesics, anesthetics and anti-inflammatory drugs, which may be mixed or eluted into the bio-material employed to fabricate implant 920 or its components. Body 922 extends from a first end 926 to a second end 928, and includes a pointed tip 930. Body 922 has an outer surface 924, which includes a first load surface 934 and a second load surface 936, and adjacent side surfaces 938. Load surfaces 934, 936 each have a wide load surface area relative to side surfaces 938. First load surface 934 includes a locking element, such as, for example, a keel 952 extending therealong. Keel 952 is serrated and configured for penetrating engagement with an articular surface of an SI joint to fix implant 920 with the articular surface.

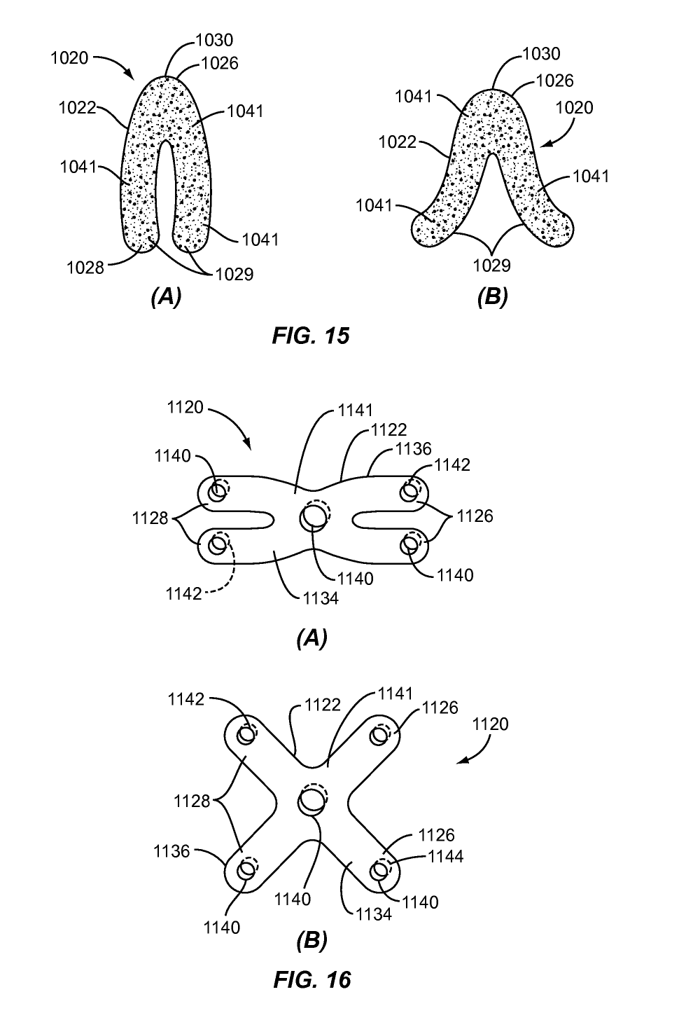

Referring to FIG. 15, in one embodiment similar to implant 20 and the implant system described above, a sacro-iliac implant 1020 is configured, for example, to treat S-I joint disorders. Sacro-iliac 1020 includes a body 1022 formed of a superelastic metallic alloy (e.g. Nitinol, super elasto-plastic metals, such as GUM METAL® manufactured by Toyotsu Material Incorporated of Japan) and includes an agent 1041 homogenously distributed thereabout, similar to that described with regard to FIG. 14. Body 1022 has a wedge-shaped configuration and extends from a first end 1026 to a second end 1028. First end 1026 has a pointed tip 1030. Second end 1028 includes arms 1029 extending from body 1022. Body 1022 is initially disposed in an unstressed orientation (15B) with arms 1029 flared outward. Body 1022 is manipulated with an externally applied force such that arms 1029 are compressed (15A) into a linear orientation profile for insertion through a reduced size opening in SI joint tissues. Body 1022 is then delivered to a joint space via a protected passageway, similar to that described above. Upon delivery of body 1022 to the joint space, the external force is removed such that arms 1029 expand to engage articular surface(s) of a SI joint for disposal with the S-I joint due to the shape memory configuration. This configuration allows body 1022 to expand to its footprint for improved stabilization and support of the SI joint while being resistant to migration or expulsion. It is envisioned that body 1022 may be fabricated from elastomeric materials such as polyurethane, silicone or PEEK.

Referring to FIG. 16, in one embodiment similar to implant 20 and the implant system described above, a sacro-iliac implant 1120 is configured, for example, to treat S-I joint disorders. Sacro-iliac implant 1120 includes a body 1122 formed of a superelastic metallic alloy (e.g. Nitinol, super elasto-plastic metals, such as GUM METAL® manufactured by Toyotsu Material Incorporated of Japan). Body 1122 has first arms 1126 and second arms 1128 extending in opposing directions from body 1122. Body 112 is initially disposed in an unstressed orientation (16B) with arms 1126, 1128 flared outward. Body 1122 is manipulated with an externally applied force such that arms 1126, 1128 are compressed (16A) into a linear orientation profile for insertion through a reduced size opening in SI joint tissues. Body 1122 is then delivered to a joint space via a protected passageway, similar to that described above. Upon delivery of body 1122 to the joint space, the external force is removed such that arms 1126, 1128 expand to engage articular surface(s) of a SI joint for disposal within the S-J joint due to the shape memory configuration. Body 1122 defines a plurality of channels 1140, similar to that described, for expelling and/or eluting at least one agent therefrom. Channels 1140 are disposed with a medial portion 1141 and arms 1126, 1128. Channels 1140 define openings 1142 and openings 1144 in side surfaces 1134, 1136, respectively. This configuration allows body 1022 to expand to its footprint for improved stabilization and support of the SI joint while being resistant to migration or expulsion. It is envisioned that body 1022 may be fabricated from electrometric materials such as polyurethane, silicone or PEEK.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A sacro-iliac implant comprising:
at least one wedge-shaped body defining an outer surface configured to engage at least one articular surface of a sacro-iliac joint.

2. A sacro-iliac implant according to claim 1, wherein the outer surface of the body includes a first side surface and a second side surface, the body defining at least one cavity extending between the first side surface and the second side surface.

3. A sacro-iliac implant according to claim 2, wherein the body defines a plurality of cavities.

4. A sacro-iliac implant according to claim 2, wherein the at least one cavity is a channel configured to elute at least one agent therefrom.

5. A sacro-iliac implant according to claim 4, wherein the channel defines at least one first opening in the first side surface and at least one second opening in the second side surface, the channel including an agent reservoir disposed between the openings.

6. A sacro-iliac implant according to claim 5, wherein the at least one first opening is disposed with the body in an orientation facing a first articular surface and the at least one second opening is disposed with the body in an orientation facing a second articular surface.

7. A sacro-iliac implant according to claim 1, wherein body has an elongated tubular configuration defining an elongated channel adapted to elute an agent therefrom and the outer surface includes barbs disposed therealong.

8. A sacro-iliac implant according to claim 1, wherein the body includes a locking element adapted to engage the at least one articular surface and being configured to prevent disengagement therefrom.

9. A sacro-iliac implant according to claim 8, wherein the locking element includes barbs disposed along the outer surface.

10. A sacro-iliac implant according to claim 8, wherein the locking element includes a keel extending from the outer surface.

11. A sacro-iliac implant according to claim 10, wherein the keel is serrated.

12. A sacro-iliac implant according to claim 2, wherein the outer surface includes a load surface extending between the first side surface and the second side surface, the load surface having a surface area at least in a range of 3 to 15 times greater than a surface area of the first side surface and the second side surface.

13. A sacro-iliac implant according to claim 1, further comprising a plurality of wedge-shaped bodies.

14. A sacro-iliac implant according to claim 1, wherein the body includes a pointed tip.

15. A sacro-iliac implant according to claim 1, wherein the body is fabricated from a shape memory material.

16. An orthopedic implant comprising:
at least one wedge shaped body defining an outer surface configured to engage a first articular surface and a second opposing articular surface, the outer surface including a first side surface and a second side surface,
the body including a pointed tip and defining at least one channel extending between the side surfaces and defining at least one first opening disposed with the body in an orientation facing the first articular surface and at least one second opening disposed with the body in an orientation facing the second articular surface.

17. An orthopedic implant according to claim 16, wherein the body includes a locking element adapted to engage the articular surfaces and being configured to prevent disengagement therefrom.

18. An orthopedic implant according to claim 16, wherein the outer surface includes a load surface extending between the first side surface and the second side surface, the load surface having a surface area at least in a range of 3-15 times greater than a surface area of the first side surface and the second side surface.

19. An orthopedic implant according to claim 16, wherein the body defines a plurality of channels.

20. A sacro-iliac implant comprising:
a wedge shaped body defining an outer surface configured to engage a first articular surface and a second opposing articular surface, the outer surface including a first side surface and a second side surface,
the body including a pointed tip and defining at least one channel extending between the side surfaces,
at least one first opening being disposed with the body in an orientation facing the first articular surface and at least one second opening disposed with the body in an orientation facing the second articular surface, and
the at least one channel including an agent reservoir disposed between the openings and the at least one channel being configured to elute at least one agent from the openings.
